(19) United States
(12) Patent Application Publication
(10) Pub. No.: US 2025/0122237 A1
(43) Pub. Date: Apr. 17, 2025

(54) POLYMORPHIC SUBSTANCE OF A-DECARBURIZATION-5alpha ANDROSTANE COMPOUND

(71) Applicant: SHANGHAI AO QI MEDICAL TECHNOLOGY CO., LTD., Shanghai (CN)

(72) Inventors: Zhihua CHEN, Shanghai (CN); Congpeng FENG, Shanghai (CN); Yajun CHEN, Shanghai (CN)

(21) Appl. No.: 18/259,808

(22) PCT Filed: Dec. 30, 2021
(86) PCT No.: PCT/CN2021/143075

§ 371 Date: Jan. 22, 2024

(30) Foreign Application Priority Data
Dec. 30, 2020 (CN) 202011613724.5

(51) Int. Cl. C07J 61/00; A61K 31/56

(52) U.S. Cl. CPC C07J 61/00; A61K 31/56

(57) ABSTRACT

A polymorphic substance of a A-decarburization-5α androstane compound (ACP-2), an application thereof and a preparation method therefor are provided. Specifically, a polymorphic substance of a A-decarburization-5α androstane compound (ACP-2), a preparation method therefor and use thereof are provided.

### TECHNICAL FIELD

The present invention belongs to the field of medicinal chemistry and specifically relates to polymorphs of A-decarbonized-5α androstane compound (ACP-2), applications thereof and preparation method therefor.

### BACKGROUND

A-decarbonized-5α androstane compounds (ACP) are brand new compound independently developed and synthesized by Rui-Lin Li et al. in 2000, and animal efficacy tests have shown that it has a good therapeutic effect on prostate hyperplasia. In further studies, it was found that A-decarbonized-5α androstane compounds have significant in vivo and in vitro anti-malignant activity, inhibiting tumor proliferation while having the advantage of alleviating weight loss of animals. They selectively prevent the division of tumor cells without affecting normal cells, thus inhibiting the proliferation of tumor cells.

It is well known that most compounds have polymorphs. The phenomenon of polymorph is not only controlled by the spatial structure and functional group properties of the molecule itself, intramolecular and intermolecular interactions and other intrinsic factors, but it is also affected by the drug synthesis process, crystallization and purification conditions, the choice of preparation excipients, preparation process and granulation methods, as well as storage conditions and other factors. The melting point, solubility, dissolution properties, chemical stability, reactivity, mechanical stability and other properties of different crystal forms may be different, and these physicochemical properties or processable properties sometimes directly affect the safety of the drug, and effective performance. Therefore, the study and control of crystal form has become an important research element in the process of drug discovery and development.

ACP-2, as a potential drug for anti-tumor and anti-prostatic hyperplasia, has not been reported with its polymorphs data. The present invention reports three new crystalline forms of ACP-2: crystal form I, crystal form II, and crystal form III by studying the nucleation mode and crystallization conditions of ACP-2. It is found that the new crystal forms have high crystallinity, good stability, low hygroscopicity, and are simple to prepare, thus facilitating the process treatment and improvement of the physical and chemical properties of the drug, improving the druggability, and facilitating large-scale production. Therefore, there is an urgent need in the field to develop polymorphs of the A-decarbonized-5α androstane compound (ACP-2), which requires simple preparing method, good stability, low hygroscopicity, and can be produced in a large scale.

### SUMMARY

The purpose of the present invention is to provide polymorphs of an A-decarbonized-5α androstane compound (ACP-2), applications thereof and preparation method therefor.

In a first aspect of the present invention, provided are crystals of a 2α, 17α-bis-ethynyl-A-decarbonized-5α-androstane-2β, 17β-bis-hydroxybispropionate compound shown in Formula I,

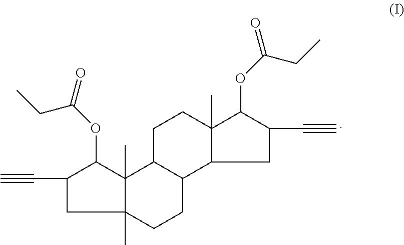 [SMILES: C#CC1CC2C3CCC4(C)CC(C#C)C(OC(=O)CC)C4(C)C3CCC2(C)C1OC(=O)CC]

In another preferred embodiment, the crystal is selected from the group consisting of: crystal form I, crystal form II and crystal form III.

In another preferred embodiment, the crystal is crystal form I, and the X-ray powder diffraction pattern of crystal form I comprises 3 or more 2θ values selected from the group consisting of: 7.1±0.2°, 11.7±0.2°, 13.4±0.2°, 16.0±0.2°, 17.1±0.2°, 20.9±0.2°, 21.1±0.2°, 23.4±0.2° and 28.6±0.2°.

In another preferred embodiment, the X-ray powder diffraction pattern of crystal form I comprises 6 or more 2θ values selected from the group consisting of: 7.1±0.2°, 11.7±0.2°, 13.4±0.2°, 16.0±0.2°, 17.1±0.2°, 20.9±0.2°, 21.1±0.2°, 23.4±0.2° and 28.6±0.2°.

In another preferred embodiment, the X-ray powder diffraction pattern of crystal form I comprises 3 or more 2θ values selected from the group consisting of: 7.1±0.2°, 13.4±0.2°, 16.0±0.2°, 17.1±0.2°, 20.9±0.2° and 21.1±0.2°.

In another preferred embodiment, the X-ray powder diffraction pattern of crystal form I comprises 2θ values selected from the group consisting of: 7.1±0.2°, 17.1±0.2° and 20.9±0.2°.

In another preferred embodiment, the X-ray powder diffraction pattern of crystal form I further comprises 1 or more 2θ values selected from the group consisting of: 14.0±0.2°, 14.2±0.2°, 16.6±0.2°, 16.8±0.2°, 18.0±0.2°, 18.3±0.2°, 19.3±0.2°, 21.4±0.2°, 23.7±0.2°, 26.3±0.2°, 28.3±0.2°, 28.9±0.2° and 31.1±0.2°.

In another preferred embodiment, the X-ray powder diffraction pattern of crystal form I further comprises 1 or more 2θ values selected from the group consisting of: 6.6±0.2°, 9.8±0.2°, 13.0±0.2°, 15.3±0.2°, 17.7±0.2°, 19.7±0.2°, 19.9±0.2°, 20.2±0.2°, 24.3±0.2°, 26.8±0.2°, 30.5±0.2°, 32.3±0.2°, 33.0±0.2°, 33.9±0.2°, 35.9±0.2° and 39.1±0.2°.

In another preferred embodiment, the crystal form I has 2θ(°) values selected from Table 1.

In another preferred embodiment, the X-ray powder diffraction pattern of crystal form I is substantially as characterized in FIG. 1.

In another preferred embodiment, the crystal is crystal form II, and the X-ray powder diffraction pattern of crystal form II comprises 3 or more 2θ values selected from the group consisting of: 14.0±0.2°, 15.2±0.2°, 16.1±0.2°, 16.7±0.2°, 16.9±0.2°, 17.1±0.2°, 18.3±0.2°, 20.9±0.2° and 24.2±0.2°.

In another preferred embodiment, the X-ray powder diffraction pattern of crystal form II comprises 6 or more 2θ values selected from the group consisting of: 14.0±0.2°, 15.2±0.2°, 16.1±0.2°, 16.7±0.2°, 16.9±0.2°, 17.1±0.2°, 18.3±0.2°, 20.9±0.2° and 24.2±0.2°.

In another preferred embodiment, the X-ray powder diffraction pattern of crystal form II comprises 3 or more 2θ values selected from the group consisting of: 14.0±0.2°, 15.2±0.2°, 16.9±0.2°, 17.1±0.2° and 18.3±0.2°.

In another preferred embodiment, the X-ray powder diffraction pattern of crystal form II comprises 2θ values selected from the group consisting of: 14.0±0.2°, 15.2±0.2° and 18.3±0.2°.

In another preferred embodiment, the X-ray powder diffraction pattern of crystal form II further comprises 1 or more 2θ values selected from the group consisting of: 7.0±0.2°, 11.6±0.2°, 12.9±0.2°, 13.4±0.2°, 17.7±0.2°, 22.4±0.2°, 23.0±0.2°, 23.7±0.2°, 26.8±0.2°, 28.8±0.2° and 37.2±0.2°.

In another preferred embodiment, the X-ray powder diffraction pattern of crystal form II further comprises 1 or more 2θ values selected from the group consisting of: 12.2±0.2°, 25.2±0.2°, 25.9±0.2°, 26.4±0.2°, 27.2±0.2°, 29.3±0.2°, 30.2±0.2°, 33.3±0.2°, 35.6±0.2°, 37.0±0.2°, and 37.8±0.2°.

In another preferred embodiment, the crystal form II has 2θ(°) values selected from Table 2.

In another preferred embodiment, the X-ray powder diffraction pattern of crystal form I is substantially as characterized in FIG. 2.

In another preferred embodiment, the crystal is crystal form III, and the X-ray powder diffraction pattern of crystal form III comprises 3 or more 2θ values selected from the group consisting of: 7.0±0.2°, 13.9±0.2°, 14.0±0.2°, 14.8±0.2°, 15.8±0.2°, 16.6±0.2°, 17.0±0.2°, 20.9±0.2° and 38.3±0.2°.

In another preferred embodiment, the X-ray powder diffraction pattern of crystal form III comprises 6 or more 2θ values selected from the group consisting of: 7.0±0.2°, 13.9±0.2°, 14.0±0.2°, 14.8±0.2°, 15.8±0.2°, 16.6±0.2°, 17.0±0.2°, 20.9±0.2° and 38.3±0.2°.

In another preferred embodiment, the X-ray powder diffraction pattern of crystal form III comprises 3 or more 2θ values selected from the group consisting of: 7.0±0.2°, 13.9±0.2°, 14.0±0.2°, 17.0±0.2° and 38.3±0.2°.

In another preferred embodiment, the X-ray powder diffraction pattern of crystal form III comprises 2θ values selected from the group consisting of: 7.0±0.2°, 17.0±0.2° and 38.3±0.2°.

In another preferred embodiment, the X-ray powder diffraction pattern of crystal form III further comprises 1 or more 2θ values selected from the group consisting of: 11.5±0.2°, 13.2±0.2°, 13.4±0.2°, 15.2±0.2°, 16.0±0.2°, 17.6±0.2°, 18.2±0.2°, 20.6±0.2°, 23.9±0.2°, 30.4±0.2°, 30.6±0.2°, 30.9±0.2° and 38.9±0.2°.

In another preferred embodiment, the X-ray powder diffraction pattern of crystal form III further comprises 1 or more 2θ values selected from the group consisting of: 6.6±0.2°, 19.0±0.2°, 23.4±0.2°, 23.6±0.2°, 26.3±0.2°, 26.8±0.2°, 28.3±0.2°, 28.7±0.2°, 30.1±0.2° and 34.4±0.2°.

In another preferred embodiment, the crystal form III has 2θ(°) values selected from Table 3.

In another preferred embodiment, the X-ray powder diffraction pattern of crystal form III is substantially as characterized in FIG. 3.

In a second aspect of the present invention, provided is a method for preparing the crystal as described in the first aspect of the present invention, wherein the crystal is crystal form I, and the method comprises steps of:
a) providing a first solution of a compound of formula I in a first solvent, wherein the first solvent is alkane, petroleum ether or a combination thereof, preferably C5-C10 alkane; and
(b) subjecting the first solution to a devitrification treatment so as to form a crystal form as described in the first aspect of the present invention, i.e., crystal form I.

In another preferred embodiment, the first solvent is selected from the group consisting of: n-hexane, n-heptane, cyclopentane, cyclohexane, petroleum ether, and combinations thereof. Some specific solvents can be listed.

In another preferred embodiment, the step (a) further comprises: dissolving the compound of formula I in the first solvent.

In another preferred embodiment, the dissolving is carried out under heating conditions with a heating temperature of 30-120° C., preferably 50-100° C.

In another preferred embodiment, for each gram of compound of formula I, the first solvent is used in a volume of 1-10 mL, preferably 4-8 mL.

In another preferred embodiment, the devitrification treatment comprises: cooling, standing, volatilizing, or a combination thereof.

In another preferred embodiment, after step (b), the method further comprises: (c) isolating the crystal form I from the solution of the previous step.

In another preferred embodiment, after step (b), the method further comprises: (d) drying the isolated crystal form I.

In a third aspect of the present invention, provided is a method for preparing the crystal as described in the first aspect of the present invention, wherein the crystal is crystal form II, and the method comprises steps of:
(i) providing a second solution of a compound of formula I in a second solvent, wherein the second solvent is a benzene solvent, preferably benzene, toluene, ethylbenzene, xylene; and
(ii) subjecting the second solution to a devitrification treatment so as to form a crystal as described in the first aspect of the present invention, i.e., crystal form II.

In another preferred embodiment, the second solvent is selected from the group consisting of toluene, ethylbenzene, xylene, and combinations thereof.

In another preferred embodiment, the step (i) further comprises: dissolving the compound of formula I in the second solvent.

In another preferred embodiment, the dissolving is carried out under heating conditions with a heating temperature of 30-120° C., preferably 40-110° C.

In another preferred embodiment, for each gram of compound of formula I, the second solvent is used in a volume of 0.5-10 mL, preferably 1-5 mL.

In another preferred embodiment, the devitrification treatment comprises: cooling, standing, volatilizing, or a combination thereof.

In another preferred embodiment, after step (ii), the method further comprises: (iii) isolating the crystal form II from the solution of the previous step.

In another preferred embodiment, after step (ii), the method further comprises: (iv) drying the isolated crystal form II.

In a fourth aspect of the present invention, provided is a method for preparing the crystal as described in the first aspect of the present invention, wherein the crystal is crystal form III, and the method comprises steps of:
(1) providing a third solution of a compound of formula I in a third solvent, wherein the third solvent is acetonitrile; and
(2) subjecting the third solution to a devitrification treatment so as to form a crystal as described in the first aspect of the present invention, i.e., crystal form III.

In another preferred embodiment, the step (1) further comprises: dissolving the compound of formula I in the third solvent.

In another preferred embodiment, the dissolving is carried out under heating conditions with a heating temperature of 30-120° C., preferably 40-90° C.

In another preferred embodiment, for each gram of compound of formula I, the third solvent is used in a volume of 1-10 mL, preferably 3-7 mL.

In another preferred embodiment, the devitrification treatment comprises: cooling, standing, volatilizing, or a combination thereof.

In another preferred embodiment, after step (2), the method further comprises: (3) isolating the crystal form III from the solution of the previous step.

In another preferred embodiment, after step (2), the method further comprises: (4) drying the isolated crystal form III.

In a fifth aspect of the invention, provided is a pharmaceutical composition, comprising:
(a) a crystal according to the first aspect of the present invention, and (b) pharmaceutically acceptable carriers.

In another preferred embodiment, dosage form of the pharmaceutical composition comprises sustained release dosage forms, or non-sustained release dosage forms.

In another preferred embodiment, the pharmaceutical composition has a dosage form of an oral dosage form, or an injectable dosage form.

In another preferred embodiment, the oral dosage form comprises tablets, capsules, films, granules.

In another preferred embodiment, the pharmaceutical composition further comprises other pharmaceutically active ingredients, more preferably, an active ingredient for the treatment of cancer, such as cisplatin, paclitaxel, or an anti-tumor antibody, etc.,

In another preferred embodiment, the total content of the crystal is from 1 to 99 wt %, preferably from 5 to 90 wt % of the pharmaceutical composition.

In a sixth aspect of the present invention, provided is a use of a crystal as described in the first aspect of the present invention or a pharmaceutical composition as described in the fifth aspect of the present invention in (I) the preparation of a medicament for preventing and/or treating tumors; and (II) the preparation of a medicament for treating prostatic hyperplasia.

In another preferred embodiment, the tumors are selected from the group consisting of: non-small cell lung cancer, small cell lung cancer, lung adenocarcinoma, squamous lung cancer, pancreatic cancer, breast cancer, prostate cancer, liver cancer, skin cancer, epithelial cell cancer, gastrointestinal mesenchymal stromal tumors, leukemia, histiocytic lymphoma, nasopharyngeal carcinoma, head and neck tumors, colon cancer, rectal cancer, bladder cancer, and gliomas.

In another preferred embodiment, the tumors are selected from the group consisting of: non-small cell lung cancer, small cell lung cancer, lung adenocarcinoma, lung squamous cancer, pancreatic cancer, breast cancer, prostate cancer, and liver cancer.

It should be understood that within the scope of the present invention, the above-mentioned technical features of the present invention and the technical features specifically described in the following (such as embodiments) can be combined with each other to form a new or preferred technical solution. Limited by space, it will not be repeated here.

### DESCRIPTION OF THE DRAWINGS

FIG. 1 shows a XRPD pattern of crystal form I.

FIG. 2 shows a XRPD pattern of crystal form II.

FIG. 3 shows a XRPD pattern of crystal form III.

FIG. 4 shows a photograph of the dried crystal form I.

FIG. 5 shows a photograph of the dried crystal form II.

FIG. 6 shows a photograph of the dried crystal form III.

### DETAILED DESCRIPTION OF EMBODIMENTS

Through extensive and in-depth research, the inventor unexpectedly discovered for the first time that polymorphs of an A-decarbonized-5α androstane compound (ACP-2), applications thereof and preparation method therefor. The polymorphs have high crystallinity, good stability and low hygroscopicity, and are easy to prepare. It is suitable for use in the preparation of pharmaceutical compositions for preventing and/or treating tumors, and for treating prostate hyperplasia. In addition, the preparation method of polymorphs of the present invention is simple and suitable for large-scale industrialized production. On this basis, the present invention is completed.

### Terms

Unless otherwise defined, all technical and scientific terms used herein have the same meanings as commonly understood by those skilled in the art.

As used herein, when used in reference to a specific value, the term “about” means that the value can vary from the enumerated value by no more than 1%. For example, as used herein, the expression “about 100” includes all values between 99 and 101 (e.g., 99.1, 99.2, 99.3, 99.4, etc.).

As used herein, the terms “contain” or “include (comprise)” may be open-ended, semi-closed, and closed-ended. In other words, the term also includes “consisting essentially of”, or “consisting of”.

As used herein, the term “n or more 20 values selected from the group consisting of” refers to including n and any positive integer greater than n (e. g., n, n+1, . . . ), where the upper limit Nup is the number of all 20 peaks value in the group. For example, “1 or more” not only includes each positive integer of 1, 2, 3, 4, 5, 6, 7, 8, 9, 10, 11, 12, 13, 14, 15, 16, 17, 18, 19, 20, 21, . . . and upper limit Nup, it also includes ranges of “2 or more”, “3 or more”, “4 or more”, “5 or more”, “6 or more”, “7 or more”, “8 or more”, “9 or more”, “10 or more”, etc., For example, “3 or more” not only includes each positive integer of 3, 4, 5, 6, 7, 8, 9, 10, 11, 12, 13, 14, 15, 16, 17, 18, 19, 20, 21, . . . and upper limit Nup, but also includes ranges of “4 or more”, “5 or more”, “6 or more”, “7 or more”, “8 or more”, “9 or more”, “10 or more”, etc.

### Compound of Formula I

The compound of the present invention is the compound of Formula I, i.e., 2α, 17α-bis-ethynyl-A-decarbonized-5α-androstane-2β, 17β-bis-hydroxybispropionate, with the structure as shown below:

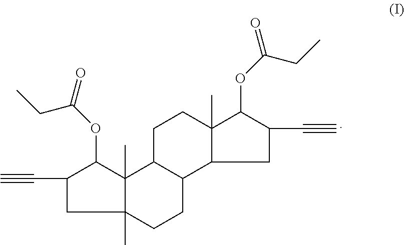 [SMILES: C#CC1CC2C3CCC4(C)CC(C#C)C(OC(=O)CC)C4(C)C3CCC2(C)C1OC(=O)CC]

This A-decarbonized-5α-androstane compound (ACP) has a good therapeutic effect on prostate hyperplasia and also has significant in vivo and in vitro anti-malignant activity, inhibiting tumor proliferation while having the advantage of alleviating the weight loss of animals. It selectively prevents the division of tumor cells without affecting normal cells, thus inhibiting the spread of tumor cells.

### Polymorph

Solid is either in amorphous form or in crystal form. In the case of crystal form, the molecule is located in the three-dimensional lattice position. When a compound is crystallized from a solution or slurry, it can be crystallized in different spatial arrays (this property is called “polymorphic phenomenon”) to form crystals with different crystalline forms. The various crystalline forms are called “polymorphs”. Different polymorphs of a given substance may differ from each other in one or more physical properties, such as solubility and dissolution rate, true specific gravity, crystal form, stacking mode, fluidity, and/or solid state stability.

The crystal of the present invention comprises a crystal selected from the group consisting of: crystal form I, crystal form II and crystal form III.

### Crystallization

The solution can be manipulated so that the solubility limit of the compound of interest is exceeded, thereby completing production-scale crystallization. This can be done by various methods, for example, dissolving the compound at a relatively high temperature, and then cooling the solution to below the saturation limit. Or by reducing the volume of liquid by boiling, atmospheric evaporation, vacuum drying or other methods. The solubility of the compound of interest can be reduced by adding an anti-solvent or a solvent in which the compound has a lower solubility or a mixture of such solvents. Another alternative method is to adjust the pH to reduce solubility. For a detailed description of crystallization please see Crystallization, 3rd edition, J W Mullens, Butterworth-Heineman Ltd. 1993, ISBN 0750611294.

If the formation of the salt is expected to occur at the same time as crystallization, and if the solubility of the salt in the reaction medium is lower than the raw material, the addition of an appropriate acid or base can lead to direct crystallization of the desired salt. Similarly, in a medium where the final desired form is less soluble than the reactant, the completion of the synthesis reaction allows the final product to crystallize directly.

The optimization of crystallization may include seeding in the crystallization medium with the desired form of crystal. In addition, many crystallization methods use a combination of the above strategies. One embodiment is to dissolve the compound of interest in a solvent at an elevated temperature, followed by adding an appropriate volume of anti-solvent in a controlled manner so that the system is just below the saturation level. At this time, adding the seed crystal in required form (and the integrity of the seed crystal is maintained), then cooling the system to complete crystallization.

### Solvate

During the contact between a compound or drug molecule and a solvent molecule, it is difficult to avoid the formation of eutectic crystal between the solvent molecule and the compound molecule and its residue in the solid substance as a result of both external and internal conditions. The substance formed after crystallization of the drug with the solvent is called solvate. The types of solvents that readily form solvates with organic compounds are water, methanol, benzene, ethanol, ether, aromatics, heterocyclic aromatic hydrocarbons, etc.

Hydrate is a special type of solvate. In the pharmaceutical industry, both in the synthesis of APIs, pharmaceutical formulations, drug storage and drug activity evaluation, hydrates have a separate discussion value because of their special characteristics.

### Pharmaceutical Composition

The present invention provides a pharmaceutical composition comprising (a) the crystal form according to the first aspect of the present invention, and (b) pharmaceutically acceptable carriers.

The “active ingredient” or “active compound” in the pharmaceutical compositions described herein refers to the compound of formula I described herein, in particular the compound of formula I in the crystal form of the invention.

The “active ingredients” or “active compounds” and pharmaceutical compositions described herein may be used for the prevention and/or treatment of tumors, prostatic hyperplasia.

The “safe and effective amount” refers to that: the amount of active ingredients is sufficient to significantly improve the condition without causing serious side effects.

Typically, the pharmaceutical composition contains 1-2,000 mg of active ingredient/dose, more preferably 10-200 mg of active ingredient/dose. Preferably, the “one dose” is a pill or an injection.

“Pharmaceutically acceptable carrier” refers to one or more compatible solid or liquid fillers or gel substances, which are suitable for human use and must have sufficient purity and low toxicity.

“Compatibility” herein refers to the ability of components of a composition to blend with each other and with the active ingredient of the invention without significantly reducing the efficacy of the active ingredient.

Examples of pharmaceutically acceptable carriers include cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oil (such as soybean oil, sesame oil, peanut oil, olive oil, etc.), polyols (such as propylene glycol, glycerin, mannitol, sorbitol, etc.), emulsifier (such as Tween®), wetting agents (such as sodium dodecyl sulfate), colorants, flavoring agents, stabilizers, antioxidants, preservatives, non-thermal raw water, etc.

In another preferred embodiment, the compound of formula I of the present invention may form a complex with a macromolecular compound or a polymer by non-bonding interaction.

In another preferred embodiment, the compound of formula I of the present invention as a small molecule may be attached to a macromolecular compound or polymer by a chemical bond. The macromolecular compound may be a biomacromolecule such as polysaccharide, protein, nucleic acid, polypeptide, etc.

There is no particular limitation on the mode of administration of the active ingredient or pharmaceutical composition of the present invention, representative modes of administration include, but are not limited to: oral, intratumoral, rectal, parenteral (intravenous, intramuscular or subcutaneous), etc.

Solid dosage forms for oral administration include capsules, tablets, pills, powders and granules.

In these solid dosage forms, the active ingredient is mixed with at least one conventional inert excipient or carrier, such as sodium citrate or dicalcium phosphate, or with one or more of the following ingredients:
(a) fillers or bulking agents, e.g., starch, lactose, sucrose, glucose, mannitol, and silicic acid;
(b) binders, e.g., hydroxymethyl cellulose, alginate, gelatin, polyvinylpyrrolidone, sucrose, and gum arabic;
(c) humectants, e.g., glycerin;
(d) disintegrants, e.g., agar, calcium carbonate, potato starch or tapioca starch, alginic acid, certain complex silicates, and sodium carbonate;
(e) retarder, e.g., paraffin;
(f) absorption accelerators, e.g., quaternary ammonium compound;
(g) wetting agents, e.g., cetearyl alcohol and glycerol monosterate;
(h) adsorbents, e.g., kaolin; and/or
(i) lubricants, e.g., talc, calcium stearate, magnesium stearate, solid polyethylene glycol, sodium dodecyl sulfate, or mixtures thereof.

In capsules, tablets and pills, the dosage form may also contain a buffering agent.

The solid dosage forms may also contain coatings and shell materials, such as casings and other materials well known in the art. They may contain opacifying agents and the release of active ingredient in such compositions may be released in a delayed manner in a portion of the digestive tract. Examples of embedding components that may be employed are polymeric substances and waxes.

Liquid dosage forms for oral administration include pharmaceutically acceptable emulsions, solutions, suspensions, syrups or tinctures. In addition to the active ingredients, the liquid dosage form may contain inert diluents conventionally used in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil, or mixtures thereof. In addition to these inert diluents, the composition may also contain auxiliaries such as wetting agents, emulsifiers, suspending agents, sweeteners, flavoring agents and flavors.

In addition to the active ingredient, the suspension may comprise suspending agents, such as ethoxylatedisooctadecanol, polyoxyethylene sorbitol and dehydrated sorbitol esters, microcrystalline cellulose, methanolic aluminum, agar, and any mixtures thereof.

The composition for parenteral injection may comprise physiologically acceptable sterile aqueous or anhydrous solutions, dispersions, suspensions or emulsions, and sterile powders for redissolution into sterile injectable solutions or dispersions. Suitable aqueous and non-aqueous carriers, diluents, solvents, or excipients include water, ethanol, polyols, and suitable mixtures thereof.

When using the pharmaceutical composition, a safe and effective amount of the compound of the present invention is administered to mammals in need of treatment (such as humans), wherein the dosage at the time of administration is the pharmaceutically considered effective dose, which is typically 1 to 2,000 mg per day, more preferably 20 to 500 mg per day for a 60 kg body weight human. Of course, the specific dosage should also consider the route of administration, the patient's health condition and other factors, which are within the skill range of skilled doctors.

The compound of the present invention can be administered alone or in combination with other therapeutic drugs (such as hypoglycemic drugs).

The compound of formula I of the present invention can also be combined with other drugs known to treat or improve similar conditions. When administered in combination, the mode of administration and dosage of the known drug may be unchanged, while the compound of formula I of the present invention is administered simultaneously or subsequently. When the compound of formula I is taken together with one or more other drugs, it is preferable to use a pharmaceutical combination that contains both one or several known drugs and the compound of formula I. Pharmaceutical combination also includes taking the compound of formula I with one or several other known drugs at overlapping time periods. The administered in combination also includes administered the compound of formula I of the present invention and one or more other known drugs at an overlapping time period. When the compound of formula I the present invention is administered in combination with one or more other drugs, the dose of the compound of formula I of the present invention or the known drug may be lower than when they are administered alone.

### Use

The present invention provides a use of crystal form I, crystal form II or crystal form III, or a pharmaceutical composition thereof in (I) the preparation of a medicament for preventing and/or treating tumors; and (II) the preparation of a medicament for treating prostatic hyperplasia.

When the crystal forms of the present invention are used as described above, they may be mixed with one or more pharmaceutically acceptable carriers or excipients, such as solvents, diluents, and the like, and may be administered orally in the form of tablets, pills, capsules, dispersible powders, granules, or suspensions (containing, e.g., about 0.05-5% suspending agent), syrups (containing, e.g., about 10-50% sugar), and elixirs (containing about 20-50% ethanol), or in the form of sterile injectable solutions or suspensions (containing about 0.05-5% suspending agent in an isotonic medium) for parenteral administration. For example, these pharmaceutical formulations may contain about 0.01-99%, preferably about 0.1%-90% by weight of the active ingredient mixed with carriers.

The two active ingredients or pharmaceutical composition of the present invention may be administered by conventional routes, which include (but are not limited to): intraocular, intramuscular, intraperitoneal, intravenous, subcutaneous, intradermal, oral, intratumoral, or topical administration. Preferred routes of administration include oral administration, intramuscular administration, or intravenous administration.

From the standpoint of ease of administration, preferred pharmaceutical compositions are solid compositions, particularly tablets and solid-filled or liquid-filled capsules.

In addition, the two active ingredients or drugs of the present invention may be used in combination with other drugs for the treatment of cancers (e.g., cisplatin, paclitaxel, antibodies against tumors, etc.).

The main advantages of the invention include:
(1) The crystal forms of the compound of the present invention have high crystallinity, good stability and low hygroscopicity.
(2) The preparation method of the crystal forms of the compound of the present invention is simple and suitable for large-scale industrialized production.
(2) The crystal forms of the compound of the present invention can be used in (I) the preparation of medicaments for the prevention and/or treatment of tumors; and (II) the preparation of medicaments for the treatment of prostatic hyperplasia.
(4) The present invention provides methods for preparing crystal form I, crystal form II, and crystal form III, wherein a dissolution precipitation method is used, and the methods of the present invention are easy for rapid and large-scale industrial production.

The present invention is further described below in conjunction with specific embodiments. It is to be understood that these examples are intended to illustrate the invention only and not to limit the scope of the invention. The experimental methods in the following examples that do not specify specific conditions are usually based on conventional conditions or conditions recommended by the manufacturer. Unless otherwise specified, percentages and portions are calculated by weight.

The experimental materials and reagents used in the following examples are commercially available sources unless otherwise specified. Ordinary temperature or room temperature refers to 4° C.-25° C., preferably 15-25° C.

### X-Ray Powder Diffraction (XRPD)

X-ray Powder Diffraction Instrument: PANalytical B.V., X'Pert Powder Diffractometer; Parameters for X-ray powder diffraction areas follows: Copper target (α=1.540600 Å), scanned at room temperature.
Voltage: 40 kilovolts (kv)
Current: 40 milliamperes (mA)
Scanning mode: continuous
Scanning range: 2.0 to 45.0 degrees
Step length: 0.020°
Test Temperature: 25° C.

### Example 1 Preparation of Crystal Form I

To 30 mL of hexane was added 5 g of ACP-2, heated and refluxed for 30 minutes, filtered; the filtrate was naturally cooled to about 20° C., then filtered to obtain a crystal, and then dried under vacuum at 30-60° C. to obtain crystal form I of ACP-2. or

To 20 mL of n-heptane was added 5 g of ACP-2, heated and refluxed for 30 minutes, filtered; the filtrate was cooled to room temperature and then cooled to about 0° C. and kept for 5 hours, then filtered to obtain a rod-like crystal, and then dried under vacuum at 30-60° C. to obtain crystal form I of ACP-2.

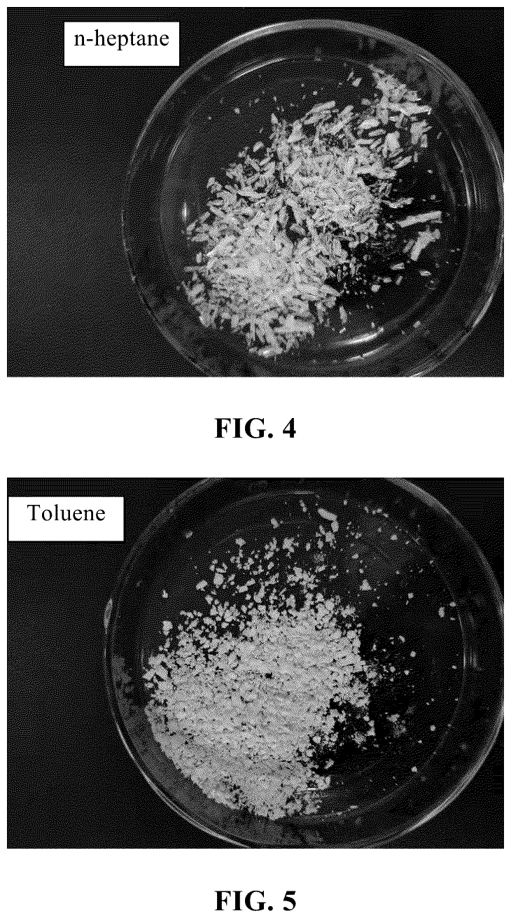

A photograph of the crystal form I prepared using n-heptane is shown in FIG. 4.

### Example 2 Crystal Form I

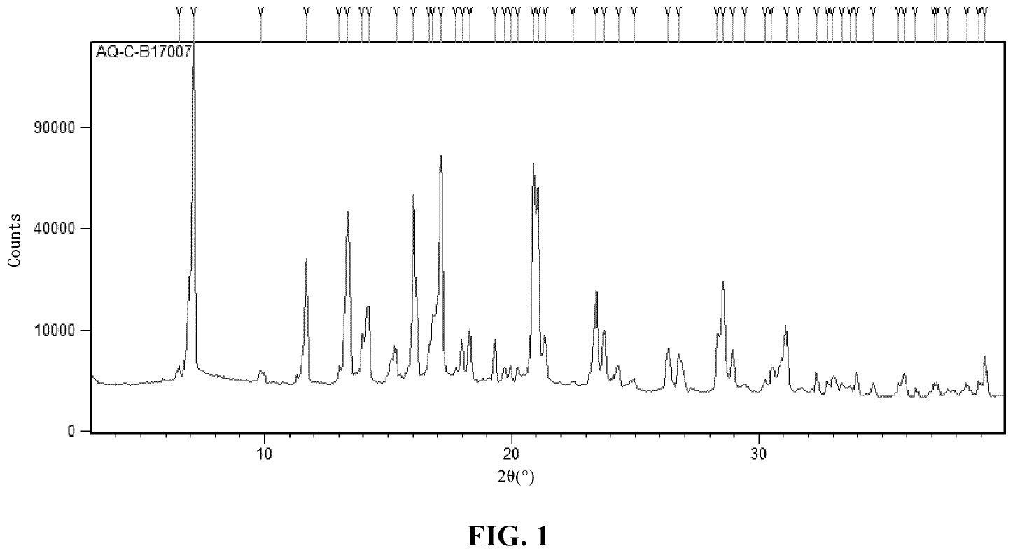

The XRD pattern of the obtained crystal form I is shown in FIG. 1 and the XRD data is basically shown in Table 1 below.

TABLE 1
XRD data of crystal form I

| | | | | Relative |
|---|---|---|---|---|
| 2θ | Height | FWHM | d-spacing(Å) | Intensity % |
| 6.6 | 1977.57 | 0.1791 | 13.4698 | 1.34 |
| 7.1 | 148083.1 | 0.0768 | 12.39572 | 100 |
| 9.8 | 1533.41 | 0.0768 | 8.99397 | 1.04 |
| 11.7 | 27384.95 | 0.1023 | 7.56788 | 18.49 |
| 13.0 | 2314.18 | 0.0512 | 6.8005 | 1.56 |
| 13.4 | 45456.66 | 0.1535 | 6.62402 | 30.7 |
| 14.0 | 7273.8 | 0.0512 | 6.34639 | 4.91 |
| 14.2 | 13418.52 | 0.1791 | 6.23073 | 9.06 |
| 15.3 | 4352.32 | 0.1279 | 5.78393 | 2.94 |
| 16.0 | 52768.55 | 0.1023 | 5.52631 | 35.63 |
| 16.6 | 4950.1 | 0.0512 | 5.32693 | 3.34 |
| 16.8 | 11251.96 | 0.0768 | 5.27547 | 7.6 |
| 17.1 | 74325.61 | 0.1279 | 5.17168 | 50.19 |
| 17.7 | 2151.69 | 0.0768 | 5.00067 | 1.45 |
| 18.0 | 6257.26 | 0.1023 | 4.93035 | 4.23 |
| 18.3 | 8790.81 | 0.1023 | 4.84658 | 5.94 |
| 19.3 | 6452.28 | 0.1023 | 4.5954 | 4.36 |
| 19.7 | 2240.52 | 0.1279 | 4.50128 | 1.51 |
| 19.9 | 2395.08 | 0.0768 | 4.45197 | 1.62 |
| 20.2 | 2180.57 | 0.0768 | 4.38723 | 1.47 |
| 20.9 | 63940.96 | 0.1023 | 4.25529 | 43.18 |
| 21.1 | 54654.88 | 0.1023 | 4.21856 | 36.91 |
| 21.4 | 7089.93 | 0.1023 | 4.16075 | 4.79 |
| 22.5 | 757.89 | 0.1535 | 3.95132 | 0.51 |
| 23.4 | 17906.11 | 0.1023 | 3.79703 | 12.09 |
| 23.7 | 8215.04 | 0.1279 | 3.74794 | 5.55 |
| 24.3 | 2528.34 | 0.1023 | 3.66135 | 1.71 |
| 24.9 | 1181.86 | 0.1023 | 3.56981 | 0.8 |
| 26.3 | 5166.28 | 0.1791 | 3.38435 | 3.49 |
| 26.8 | 3993.2 | 0.1279 | 3.33244 | 2.7 |
| 28.3 | 7512.33 | 0.0768 | 3.15071 | 5.07 |
| 28.6 | 20795.25 | 0.1023 | 3.12544 | 14.04 |
| 28.9 | 5072.1 | 0.1279 | 3.08528 | 3.43 |
| 29.4 | 742.24 | 0.2047 | 3.03724 | 0.5 |
| 30.3 | 1207.96 | 0.1023 | 2.95369 | 0.82 |
| 30.5 | 2562.74 | 0.1535 | 2.92981 | 1.73 |
| 31.1 | 8221.94 | 0.1279 | 2.87526 | 5.55 |
| 31.6 | 395.17 | 0.307 | 2.82951 | 0.27 |
| 32.3 | 2175.84 | 0.1023 | 2.76954 | 1.47 |
| 32.8 | 1149.11 | 0.0768 | 2.73359 | 0.78 |
| 33.0 | 1771.05 | 0.0768 | 2.71512 | 1.2 |
| 33.4 | 982.73 | 0.0768 | 2.68653 | 0.66 |
| 33.7 | 815.3 | 0.1023 | 2.66037 | 0.55 |
| 33.9 | 1964.67 | 0.1535 | 2.64202 | 1.33 |
| 34.6 | 1022.8 | 0.1279 | 2.59163 | 0.69 |
| 35.6 | 1062.9 | 0.0768 | 2.51852 | 0.72 |
| 35.9 | 2101.42 | 0.1279 | 2.50236 | 1.42 |
| 36.3 | 589.89 | 0.0768 | 2.47205 | 0.4 |
| 37.1 | 1001 | 0.0936 | 2.42236 | 0.68 |
| 37.2 | 1121.82 | 0.0768 | 2.41671 | 0.76 |
| 37.6 | 550.74 | 0.0768 | 2.39004 | 0.37 |
| 38.4 | 888.64 | 0.2558 | 2.34336 | 0.6 |
| 38.9 | 1308.21 | 0.0768 | 2.31587 | 0.88 |
| 39.1 | 4264.17 | 0.0768 | 2.30228 | 2.88 |

### Example 3 Preparation of Crystal Form II

To 10 mL of toluene was added 3 g of ACP-2, heated to reflux until solids were dissolved, and then filtered; the filtrate was naturally cooled to about 20° C., then filtered to obtain a tabular crystal, and then dried under vacuum at 30-60° C. to obtain crystal form II of ACP-2.

A photograph of the dried crystal form II is shown in FIG. 5.

### Example 4 Crystal Form II

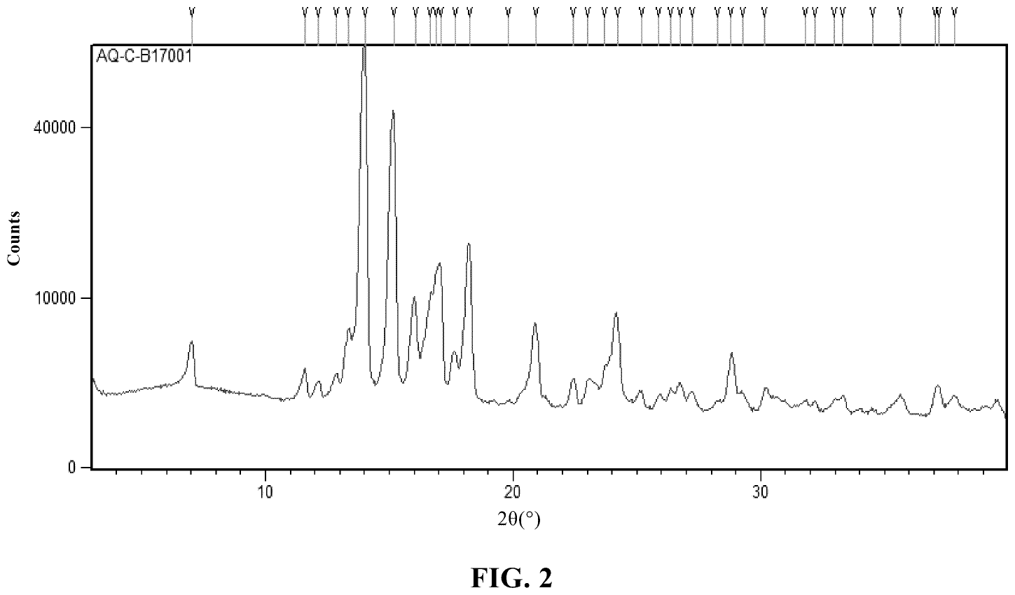

The XRD pattern of the obtained crystal form II is shown in FIG. 2, and the XRD data is basically shown in Table 2 below.

TABLE 2
XRD data of crystal form II

| | | | | Relative |
|---|---|---|---|---|
| 2θ | Height | FWHM | d-spacing(Å) | Intensity % |
| 7.0 | 3278.32 | 0.1791 | 12.54268 | 6.01 |
| 11.6 | 1679.97 | 0.1279 | 7.62353 | 3.08 |
| 12.2 | 973.71 | 0.2047 | 7.27767 | 1.79 |
| 12.9 | 1503.18 | 0.1535 | 6.87132 | 2.76 |
| 13.4 | 5193.01 | 0.2047 | 6.62885 | 9.52 |
| 14.0 | 54522.28 | 0.2814 | 6.30762 | 100 |
| 15.2 | 38935.59 | 0.2558 | 5.82841 | 71.41 |
| 16.1 | 8171.55 | 0.2558 | 5.51875 | 14.99 |
| 16.7 | 8918.19 | 0.1535 | 5.32004 | 16.36 |
| 16.9 | 11774.07 | 0.1279 | 5.24838 | 21.59 |
| 17.1 | 11727.27 | 0.1535 | 5.18826 | 21.51 |
| 17.7 | 3218.62 | 0.2303 | 5.02324 | 5.9 |
| 18.3 | 15249.17 | 0.2558 | 4.85733 | 27.97 |
| 19.8 | 194.95 | 0.1535 | 4.48564 | 0.36 |
| 20.9 | 5696.74 | 0.2558 | 4.24266 | 10.45 |
| 22.4 | 1415.24 | 0.2303 | 3.96323 | 2.6 |
| 23.0 | 1397.39 | 0.1791 | 3.86306 | 2.56 |
| 23.7 | 2246.19 | 0.1279 | 3.75233 | 4.12 |
| 24.2 | 6585 | 0.2558 | 3.6758 | 12.08 |
| 25.2 | 748.6 | 0.2047 | 3.53261 | 1.37 |
| 25.9 | 622.33 | 0.2047 | 3.43916 | 1.14 |
| 26.4 | 962 | 0.1023 | 3.38139 | 1.76 |
| 26.8 | 1312.97 | 0.2303 | 3.32932 | 2.41 |
| 27.2 | 766.31 | 0.1791 | 3.27513 | 1.41 |
| 28.2 | 419.78 | 0.2047 | 3.1598 | 0.77 |
| 28.8 | 3297.63 | 0.2558 | 3.0995 | 6.05 |
| 29.3 | 787.28 | 0.1535 | 3.04869 | 1.44 |
| 30.2 | 1069.14 | 0.2303 | 2.9612 | 1.96 |
| 31.8 | 497.06 | 0.2047 | 2.81291 | 0.91 |
| 32.2 | 450.51 | 0.1535 | 2.78045 | 0.83 |
| 33.0 | 532.33 | 0.1535 | 2.71649 | 0.98 |
| 33.3 | 743.97 | 0.2047 | 2.68794 | 1.36 |
| 34.5 | 167.29 | 0.2047 | 2.59912 | 0.31 |
| 35.6 | 874.63 | 0.1535 | 2.51914 | 1.6 |
| 37.0 | 1048.78 | 0.0936 | 2.42503 | 1.92 |
| 37.2 | 1247.1 | 0.1791 | 2.41735 | 2.29 |
| 37.8 | 689.39 | 0.307 | 2.37721 | 1.26 |

### Example 5 Preparation of Crystal Form III

To 10 mL of acetonitrile was added 2 g of ACP-2, heated at 70° C. until solids were dissolved, and filtered; the filtrate was cooled to 20° C. and then cooled to about 0° C. and kept for 5 hours, then filtered to obtain a rod-like crystal, and then dried under vacuum at 30-60° C. to obtain crystal form III of ACP-2.

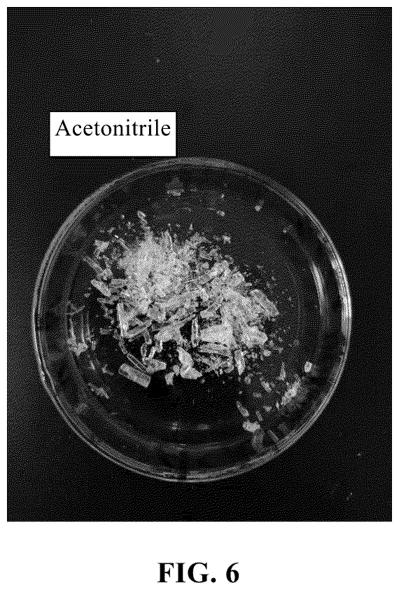

A photograph of the dried crystal form III is shown in FIG. 6.

### Example 6 Crystal Form III

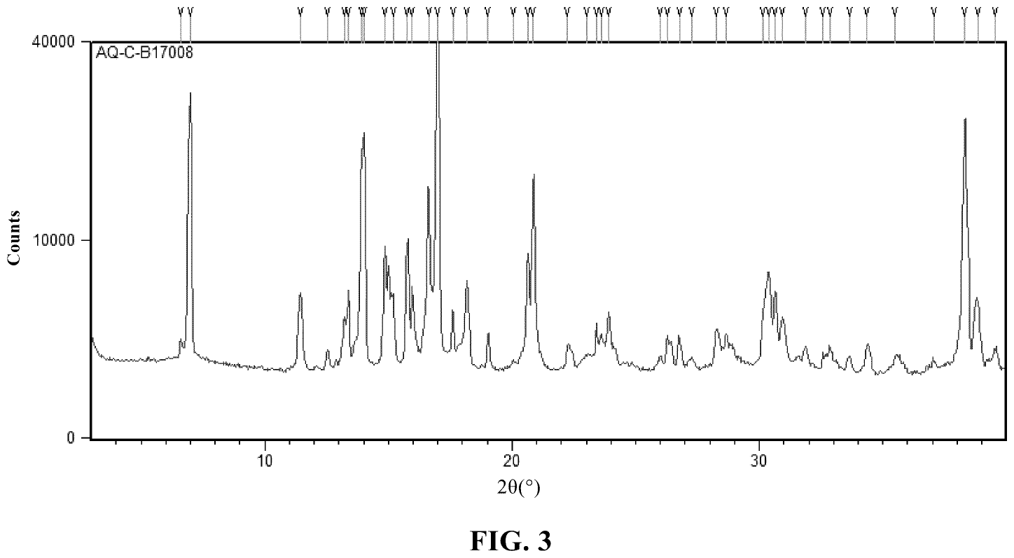

The XRD pattern of the obtained crystal form III is shown in FIG. 3, and the XRD data is basically shown in Table 3 below.

TABLE 3
XRD data of crystal form III

| | | | | Relative |
|---|---|---|---|---|
| 2θ | Height | FWHM | d-spacing(Å) | Intensity % |
| 6.6 | 1009.34 | 0.0768 | 13.38747 | 1.01 |
| 7.0 | 29895.66 | 0.1535 | 12.61826 | 29.83 |
| 11.5 | 4134.31 | 0.2047 | 7.72272 | 4.13 |
| 12.6 | 772.31 | 0.1279 | 7.0514 | 0.77 |
| 13.2 | 2535.05 | 0.0768 | 6.6969 | 2.53 |
| 13.4 | 4431.02 | 0.0768 | 6.61654 | 4.42 |
| 13.9 | 19350.8 | 0.0768 | 6.36727 | 19.31 |
| 14.0 | 22128.29 | 0.1023 | 6.30957 | 22.08 |
| 14.8 | 8097.7 | 0.1023 | 5.96681 | 8.08 |
| 15.2 | 4086.18 | 0.0768 | 5.8266 | 4.08 |
| 15.8 | 8828.93 | 0.0768 | 5.62142 | 8.81 |
| 16.0 | 4481.05 | 0.0768 | 5.55022 | 4.47 |
| 16.6 | 15358.96 | 0.0768 | 5.33384 | 15.33 |
| 17.0 | 100205.2 | 0.1023 | 5.21643 | 100 |
| 17.6 | 2967.84 | 0.0768 | 5.03578 | 2.96 |
| 18.2 | 5356.58 | 0.1023 | 4.87668 | 5.35 |
| 19.0 | 1610.71 | 0.0768 | 4.66238 | 1.61 |
| 20.0 | 345.05 | 0.1535 | 4.42876 | 0.34 |
| 20.6 | 7643.27 | 0.1023 | 4.30227 | 7.63 |
| 20.9 | 16625.63 | 0.1023 | 4.25804 | 16.59 |
| 22.3 | 987.1 | 0.1023 | 3.99472 | 0.99 |
| 23.0 | 589.21 | 0.307 | 3.86224 | 0.59 |
| 23.4 | 2088.29 | 0.0768 | 3.80083 | 2.08 |
| 23.6 | 1563.19 | 0.0768 | 3.77088 | 1.56 |
| 23.9 | 2706.71 | 0.1023 | 3.72407 | 2.7 |
| 26.0 | 617.88 | 0.1023 | 3.42842 | 0.62 |
| 26.3 | 1496.75 | 0.0768 | 3.39169 | 1.49 |
| 26.8 | 1510.14 | 0.1279 | 3.33174 | 1.51 |
| 27.3 | 448.49 | 0.2047 | 3.27214 | 0.45 |
| 28.3 | 1823.74 | 0.1535 | 3.15597 | 1.82 |
| 28.7 | 1672.98 | 0.0768 | 3.11389 | 1.67 |
| 30.1 | 2390.66 | 0.1023 | 2.96683 | 2.39 |
| 30.4 | 6007.72 | 0.1279 | 2.94209 | 6 |
| 30.6 | 4361.74 | 0.1023 | 2.91702 | 4.35 |
| 30.9 | 2608.74 | 0.1535 | 2.89165 | 2.6 |
| 31.9 | 907.46 | 0.1535 | 2.80931 | 0.91 |
| 32.6 | 632.42 | 0.0768 | 2.75044 | 0.63 |
| 32.9 | 954.85 | 0.1023 | 2.7263 | 0.95 |
| 33.7 | 543.31 | 0.2047 | 2.66291 | 0.54 |
| 34.4 | 1007.65 | 0.1279 | 2.60955 | 1.01 |
| 35.5 | 610.95 | 0.1023 | 2.52895 | 0.61 |
| 37.1 | 383.6 | 0.1535 | 2.42633 | 0.38 |
| 38.3 | 24914.25 | 0.1791 | 2.34864 | 24.86 |
| 38.9 | 3421.39 | 0.2558 | 2.31756 | 3.41 |
| 39.5 | 867.93 | 0.1535 | 2.27886 | 0.87 |

### Example 7 Stability of Crystal Form I

After 6 months of accelerated test (test conditions 40±2° C., 75%±5% RH), the results show that the crystal of the crystal form I prepared in Example 1 is very stable, and the purity of the crystal form I remained basically unchanged in comparison to the newly prepared (0 month) crystal form I, always being above 99%, with no obvious degradation impurities.

### Example 8 Stability of Crystal Form II

After 6 months of accelerated test (test conditions 40±2° C., 75%±5% RH), the results show that the crystal of the crystal form II of Example 3 is very stable, and the purity of the crystal form II remained basically unchanged in comparison to the newly prepared (0 month) crystal form II, always being above 99%, with no obvious degradation impurities.

### Example 9 Stability of Crystal Form III

After 6 months of accelerated test (test conditions 40±2° C., 75%±5% RH), the results show that the crystal of the crystal form III prepared in Example 5 is very stable, and the purity of the crystal form III remained basically unchanged in comparison to the newly prepared (0 month) crystal form III, always being above 99%, with no obvious degradation impurities.

### Example 10 Hygroscopicity Test of Polymorphs

The test was performed in accordance with the guideline for drug hygroscopicity test (Chinese Pharmacopoeia 2010 Edition, Part II, Appendix XIX J).

1. 4 dry glass weighing flasks with caps (outer diameter of 60 mm, height of 30 mm) were taken and placed in a glass desiccator with a saturated solution of ammonium sulfate placed in the lower part of a constant temperature and humidity chamber at 25° C.±1° C. (“constant temperature and humidity desiccator”) one day before the test.

2. After each empty weighing flask with its cap was placed in the “constant temperature and humidity desiccator” for 24 hours, the respective weights were precisely weighed in sets (weighing flasks+caps) and counted as m1.

3. An appropriate amount of the sample of crystal form I prepared in Example 1 was taken and placed flatly in a weighed glass weighing flask (the thickness of the sample was about 1 mm), capped, and the weights of the weighing flasks (weighing flasks+caps+samples) at this time were precisely weighed, and were counted as m2.

4. After each sample was placed in the “constant temperature and humidity desiccator” for 24 hours, the weights of each weighing flask (weighing flask+cap+sample) at this time were precisely weighed, and were counted as m3.

5. The humidity weight gain percentage of each sample was calculated (the equation is as follows), when the humidity weight gain percentage is less than 0.2%, it is defined as no or almost no hygroscopicity. When the humidity weight gain percentage is greater than or equal to 0.2%, but less than 2.0%, it is defined as slightly hygroscopicity.

$$Weight gain percentage = [ m 3 - m 2 ) / ( m 2 - m 1 ) ] \times 100 \%$$

The hygroscopicity property of crystal form I of the present invention was determined according to the above procedure, and the results showed that the weight gain percentage of crystal form I is [(36.6509−36.6492)/(36.6492−35.6515)]×100%=0.17%. It can be seen that the crystal form I has almost no hygroscopicity.

The operations of the hygroscopicity test described above were repeated, with the difference being that crystal form II prepared in Example 3 and crystal form III prepared in Example 5 were used as test samples, and it was found that both crystal form II and crystal form III of the present invention were very stable and has essentially no hygroscopicity.

Thus, the polymorphs of the present invention are very suitable for use in pharmaceutical compositions. Moreover, the polymorphs of the present invention are easy to collect, not easily wasted, and not easily dispersed during the manufacturing process of pharmaceuticals, which are helpful in defending the health of the operators.

All documents referred to in the present invention are incorporated by reference herein as if each document is individually incorporated by reference. Further, it should be understood that upon reading the above teachings of the present invention, various alterations or modifications may be made to the present invention by those skilled in the art, and those equivalents also fall within the scope defined by the appended claims of the present application.

1. A crystal of 2α,17α-bis-ethynyl-A-decarbonized-5α-androstane-2β,17β-bis-hydroxybispropionate compound of Formula I, wherein, the compound of formula I is as follows:

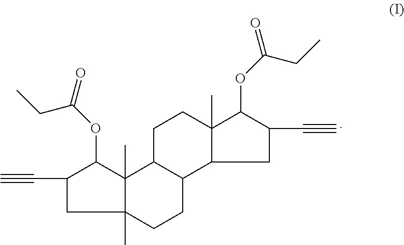 [SMILES: C#CC1CC2C3CCC4(C)CC(C#C)C(OC(=O)CC)C4(C)C3CCC2(C)C1OC(=O)CC]

2. The crystal of claim 1, wherein the crystal is selected from the group consisting of: crystal form I, crystal form II and crystal form III.

3. The crystal of claim 2, wherein the crystal is crystal form I, and the X-ray powder diffraction pattern of crystal form I comprises 3 or more 2θ values selected from the group consisting of: 7.1±0.2°, 11.7±0.2°, 13.4±0.2°, 16.0±0.2°, 17.1±0.2°, 20.9±0.2°, 21.1±0.2°, 23.4±0.2° and 28.6±0.2°.

4. The crystal of claim 2, wherein the crystal is crystal form II, and the X-ray powder diffraction pattern of crystal form II comprises 3 or more 2θ values selected from the group consisting of: 14.0±0.2°, 15.2±0.2°, 16.1±0.2°, 16.7±0.2°, 16.9±0.2°, 17.1±0.2°, 18.3±0.2°, 20.9±0.2° and 24.2±0.2°.

5. The crystal of claim 2, wherein the crystal is crystal form III, and the X-ray powder diffraction pattern of crystal form III comprises 3 or more 2θ values selected from the group consisting of: 7.0±0.2°, 13.9±0.2°, 14.0±0.2°, 14.8±0.2°, 15.8±0.2°, 16.6±0.2°, 17.0±0.2°, 20.9±0.2° and 38.3±0.2°.

6. A method for preparing the crystal as described in claim 3, wherein the crystal is crystal form I, and the method comprises steps of:
a) providing a first solution of a compound of formula I in a first solvent, wherein the first solvent is alkane, petroleum ether or a combination thereof, preferably C5-C10 alkane; and
(b) subjecting the first solution to a devitrification treatment so as to form a crystal form I.

7. A method for preparing the crystal as described in claim 4, wherein the crystal is crystal form II, and the method comprises steps of:
(i) providing a second solution of a compound of formula I in a second solvent, wherein the second solvent is a benzene solvent, preferably benzene, toluene, ethylbenzene, xylene; and
(ii) subjecting the second solution to a devitrification treatment so as to form a crystal form II.

8. A method for preparing the crystal as described in claim 5, wherein the crystal is crystal form III, and the method comprises steps of:
(i) providing a third solution of a compound of formula I in a third solvent, wherein the third solvent is acetonitrile; and
(ii) subjecting the third solution to a devitrification treatment so as to form a crystal form III.

9. A pharmaceutical composition comprising:
(a) the crystal according to claim 1, and (b) pharmaceutically acceptable carriers.

10. A use of a crystal according to claim 1 (I) the preparation of a medicament for preventing and/or treating tumors; and (II) the preparation of a medicament for treating prostatic hyperplasia.
